(19) United States
(12) Patent Application Publication
(10) Pub. No.: US 2015/0225763 A1
(43) Pub. Date: Aug. 13, 2015

(54) BIOMARKER

(71) Applicant: ISIS INNOVATION LIMITED, Oxford, Oxfordshire (GB)

(72) Inventor: Climent Casals-Pascual, Oxford (GB)

(73) Assignee: Isis Innovation Limited, Oxford (GB)

(21) Appl. No.: 14/428,935

(22) PCT Filed: Sep. 12, 2013
(86) PCT No.: PCT/GB2013/052391

§ 371 Date: Mar. 17, 2015

(30) Foreign Application Priority Data
Sep. 17, 2012 (GB) 1216562.7

(51) Int. Cl. C12Q 1/04

(52) U.S. Cl. CPC C12Q 1/04; G01N 2333/445

(57) ABSTRACT

A method for determining the malarial status of a subject, comprising the steps of: providing a biological sample obtained from a subject; and determining the level, or presence, of PF10_0121 (hypoxanthine phosphoribosyltransferase, pHPRT) and/or PF11_0208 (phosphoglycerate mutase, pPGM) in the biological sample.

The present invention relates to one or more biomarkers for determining the malarial status of a subject, and to uses of the one or more biomarkers.

Human malaria is a life-threatening disease caused by Plasmodium parasites which accounts for approximately 655,000 deaths yearly, primarily in African children. Five Plasmodium species cause human malaria, but the majority of deaths are attributable to Plasmodium falciparum. Since most deaths in children admitted to hospital with severe malaria occur within the first 24 hours, it is critical to diagnose and treat malaria patients promptly.

The standard method to diagnose malaria infection requires the visualization of the parasite in blood using light microscopy. However, laboratory facilities to diagnose malaria are not available in many resource-poor endemic settings, and the administration of antimalarials is often based on non-specific clinical symptoms. Treatment allocation based on clinical presentation results in over diagnosis of malaria and thus in the overuse of antimalarial treatment, which contributes to the increasing resistance of Plasmodium falciparum to available drugs. Consequently, the World Health Organisation recommends that all suspected malaria cases should be confirmed with a parasite-based diagnostic assay. A number of commercially available rapid diagnostic tests (RDTs) based on lateral flow immunochromatographic assays (dipsticks) that detect Plasmodium falciparum proteins have been developed and implemented with variable success. RDTs are relatively inexpensive, simple to use and results can be obtained in approximately 20 minutes.

The most widely available RDTs used in malaria detect histidine-rich protein 2 (HRP-2), lactate dehydrogenase (pLDH) and aldolase (pFBPA). Whilst these markers have value they do have limitations. For example, the HRP2 antigen may persist in blood after treatment with antimalarials for up to 33 days, which invalidates the use of this test to monitor treatment success. Similarly, the identification of HRP-2 in current and recent infections limits the diagnostic value of HRP-2 in malaria-endemic areas with high transmission intensity. Moreover, parasite populations with deletions in the histidine rich repeat region of the hrp2 gene have been documented. pLDH does not persist in blood but Plasmodium falciparum gametocytes, the sexual stage of malaria parasites that does not cause acute infection, produce pLDH and this may produce a positive test despite clearance of the asexual parasite forms that have caused the acute infection.

There therefore remains a need for additional biomarkers for malaria.

It is an aim of the present invention therefore to provide one or more biomarker(s) that may be used to give an indication of the malarial status in an individual and/or which may also be used to assess the type of treatment that is appropriate for such an individual.

In a first aspect, the present invention provides a method for determining the malarial status of a subject, comprising the steps of:
i. providing a biological sample obtained from a subject;
ii. determining the level, or presence, of PF10_{—}0121 (hypoxanthine phosphoribosyltransferase, pHPRT) and/or PF11_{—}0208 (phosphoglycerate mutase, pPGM) in the biological sample.

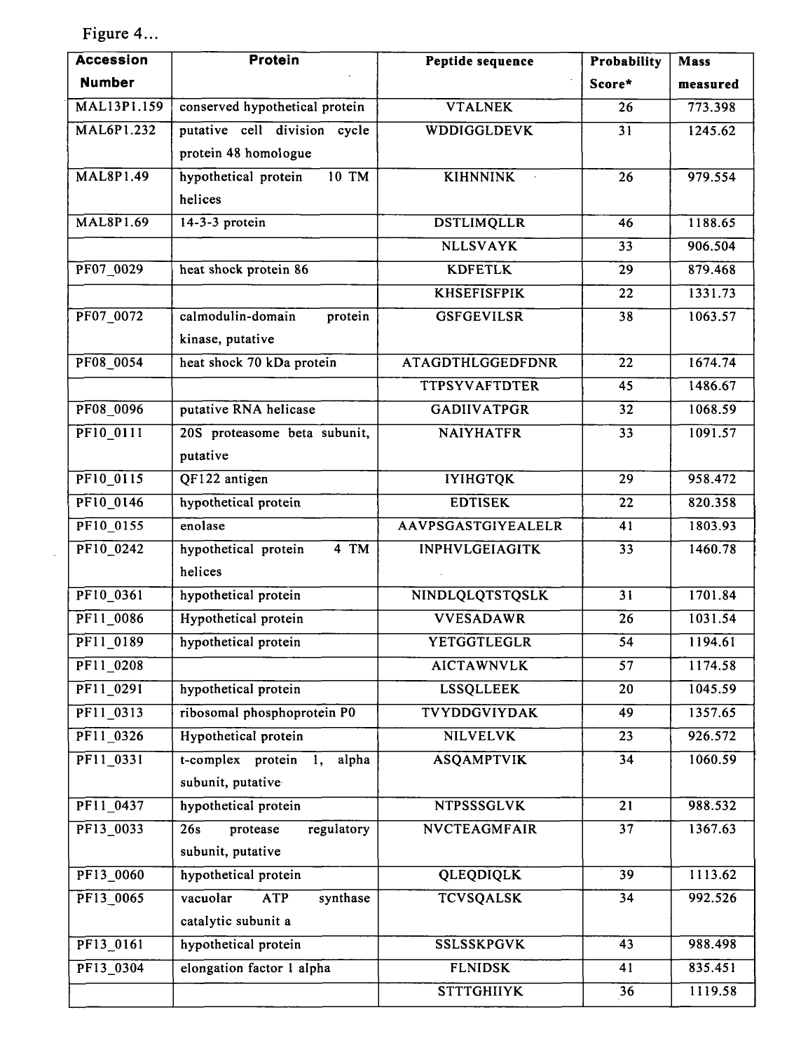

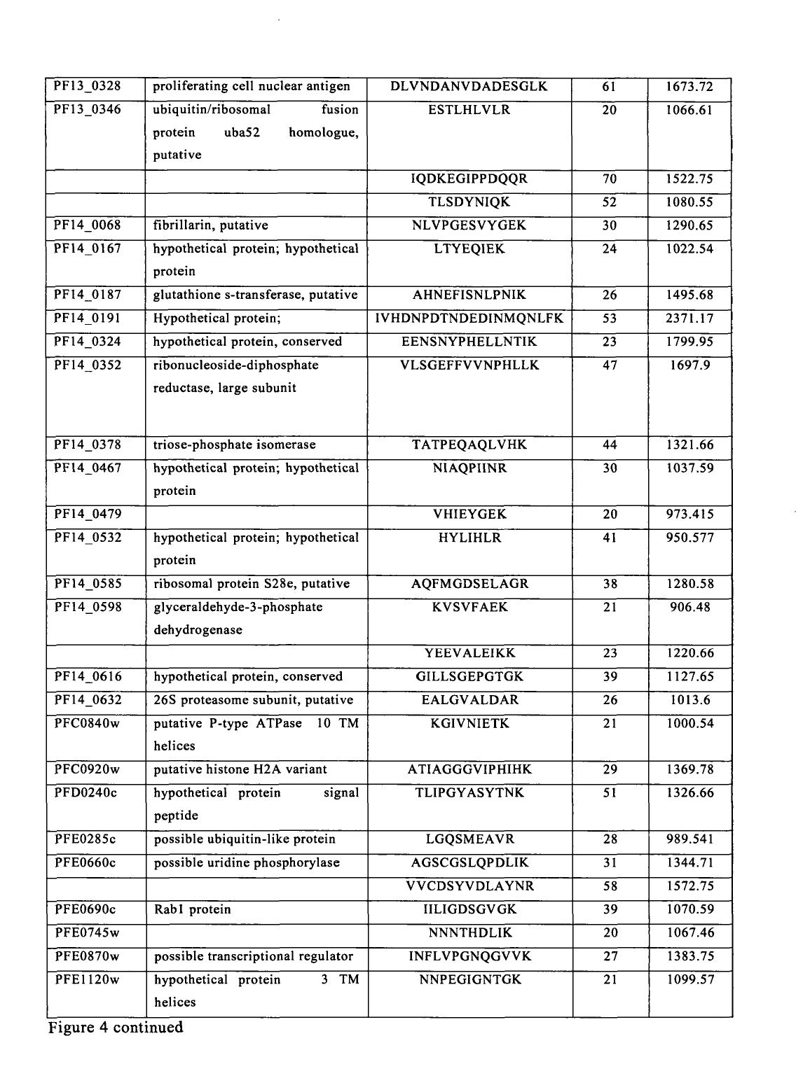

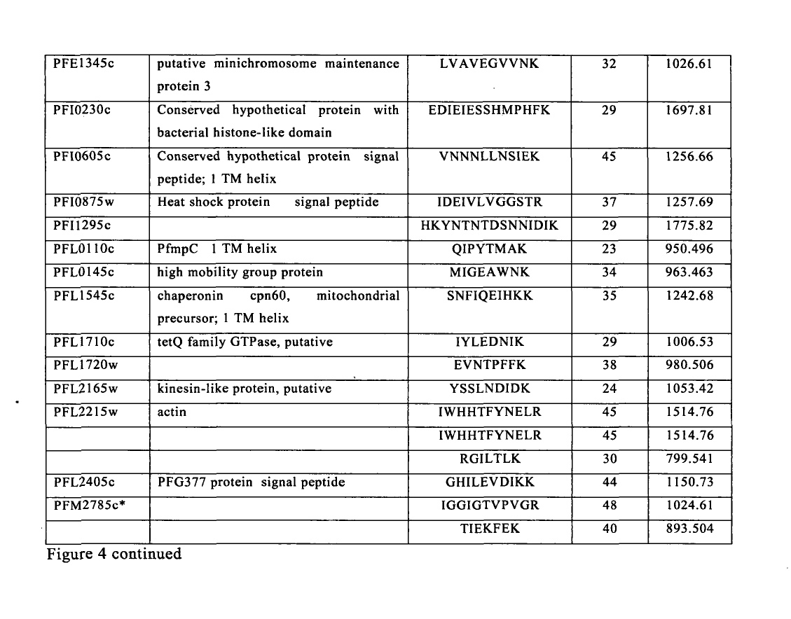

In an alternative embodiment, in step (ii) of the method of the invention the level, or presence, of one or more of the proteins identified in FIG. 4 is determined. The proteins identified in FIG. 4 may be defined by the name given in the table and/or by the accession number given in the table, the proteins are referred to herein as the “alternative biomarkers”. The alternative biomarker may be any protein identified in FIG. 4, in particular it may be any one or more of the following: 14-3-3 protein, heat shock protein 86, heat shock 70 kDa protein, QF122 antigen, enolase, ribosomal phosphoprotein P0, vacuolar ATP synthase catalytic subunit a, elongation factor 1 alpha, proliferating cell nuclear antigen, ribonucleoside-diphosphate reductase large subunit, triose-phosphate isomerise, glyceraldehyde-3-phosphate dehydrogenase, Rab1, heat shock protein signal peptide, PfmpC 1 TM helix, high mobility group protein, chaperonin cpn60 mitochondrial precursor and actin.

Preferably the presence of pHPRT and/or pPGM and/or any of the alternative biomarkers is indicative/diagnostic of malaria in a subject.

The method of the invention may include a further step of concluding a subject has malaria if pHPRT and/or pPGM and/or one or more of the alternative biomarkers are present in the sample. As both pHPRT and pPGM and the alternative biomarkers are parasite proteins they should not be present at any level in a subject who does not have malaria.

The method of the invention may further comprise the step of comparing the level determined in (ii) with one or more pre-determined reference values.

The level of pHPRT and/or pPGM and/or one or more of the alternative biomarkers may be compared to the level in a control sample, preferably a non-infected sample, to allow for any inaccuracy or background in the test method used.

In a preferred embodiment in step (ii) the level of at least pHPRT is determined

The method of the invention may allow the diagnosis of acute or symptomatic malaria, that is, the presence of pHPRT and optionally also pPGM and/or one or more of the alternative biomarkers, may allow a diagnosis of acute malaria. Wherein acute malaria is defined as the presence of malarial parasites and symptoms of malaria, for example a fever.

The step of obtaining the sample preferably does not form part of the invention

The term ‘biological sample’ refers to a sample of biological fluid obtained for the purpose of diagnosis or evaluation of a subject of interest. Preferred biological samples include, but are not limited to, blood, serum, plasma, saliva and cerebrospinal fluid. In addition, the person skilled in the art would realise that some test samples would be more readily analyzed following a fractionation or purification procedure, for example, separation of whole blood into serum or plasma components.

The biological sample may be a plasma sample obtained from a subject

The term ‘level’ as defined herein refers to the amount or concentration of pHPRT and/or pPGM and/or one or more of the alternative biomarkers contained in the biological sample.

The phrase “malarial status” includes any manifestation of malaria. For example, the presence or absence of malaria, the progression of malaria, and the effectiveness or response of a subject to a treatment for malaria.

The method of the invention may be used, for example, for any one or more of the following: to diagnose malaria; to advise on the prognosis of a subject with malaria; to monitor disease progression; and to monitor effectiveness or response of a subject to a particular treatment.

Preferably the method of the invention allows the diagnosis of malaria from the analysis of the level of pHPRT and/or pPGM and/or one or more of the alternative biomarkers in a sample from the patient. Preferably at least pHPRT levels are measured.

The level of pHPRT and/or pPGM and/or one or more of the alternative biomarkers may be compared to a reference value. Wherein the reference value be the level of expression of the same protein in a sample from one or more subjects who do not have malaria—as determined, for example, by the visualisation of the parasite light microscopy. These samples have so called “normal values” of the biomarkers.

Alternatively, the reference value may be a previous value obtained for a specific subject. This kind of reference may be used if the method is to be used to monitor progression of an infection or to monitor response of a subject to a particular treatment.

The level of pHPRT and/or pPGM and/or one or more of the alternative biomarkers may be evaluated by any suitable method. For example if protein levels are to be determined any of the group comprising immunoassays, spectrometry, western blot, ELISA, immunoprecipitation, slot or dot blot assay, isoelectric focussing, SDS-PAGE and antibody microarray immunohistological staining, radio immuno assay (RIA), fluoroimmunoassay, an immunoassay using an avidin-biotin or streptavidin-biotin system, etc and combinations thereof may be used. These methods are well known to persons skilled in the art. Other methods may also be used.

It may be sufficient to simply determine whether pHPRT and/or pPGM and/or one or more of the alternative biomarkers are present in a sample, absolute values may not be necessary.

Preferably the method of the invention allows a result to be obtained in less than 24 hours, preferably less than 12 hours, less 6 hours, less than 4 hours, less than 3 hours, less than 2 hours, less than 1 hour, more preferably less than 30 minutes.

The method of the invention may be used in conjunction with an assessment of clinical symptoms to provide a more effective diagnosis of malaria.

The present invention may also provide a method for determining the appropriate treatment for a subject comprising the steps of:
(a) providing a biological sample obtained from a subject;
(b) determining the level, or presence, of pHPRT and/or pPGM in the biological sample from said subject; and
(c) using the results in (b) to determine the most appropriate therapy.

In an alternative embodiment, in step (b) the level, or presence, of one or more of the alternative biomarkers may be determined.

For example, a patient with pHPRT and/or pPGM and/or one or more of the alternative biomarkers in the sample may be treated as having malaria and anti-malarial drugs will be administered.

The method of the invention may include a further step of comparing the level of pHPRT and/or pPGM and/or one or more of the alternative biomarkers determined in step (b) with one or more predetermined reference values prior to step (c). Preferably the level of pHPRT is determined.

In another embodiment of the invention, the method of the invention which uses pHPRT and/or pPGM and/or one or more of the alternative biomarkers to determine a diagnosis of malaria may be used in combination with the clinical features, for example fever, to determine a diagnosis of malaria.

The method of the present invention may be carried out in vitro.

The subject may be a human. In another embodiment the subject may be a mammal, for example, a dog, cat, horse, cow, monkey, ape, rodent, hamster, rat, or guinea pig.

According to another aspect of the invention, the invention provides a method of determining the response of a subject with malaria to a particular treatment comprising the steps of:
(a) providing a biological sample obtained from a subject;
(b) determining the level of pHPRT and/or pPGM in the biological sample from said subject; and
(c) comparing the level of pHPRT and/or pPGM determined in step (b) with one or more reference values.

In an alternative embodiment, in step (b) the level of one or more of the alternative biomarkers may be determined, and in step (c) the level of one or more of the alternative biomarkers is compared with a reference value.

The reference value may be the level of pHPRT and/or pPGM and/or one or more of the alternative biomarkers in an earlier sample from the same subject.

According to another aspect of the invention, the invention provides a method for treating malaria in a patient comprising: requesting a test providing the results of an analysis to determine whether the patient has pHPRT and/or pPGM in a biological sample from the patient and administering antimalarial treatment to the patient if pHRT and/or pPGM are found in the biological sample.

According to a still further aspect, the invention provides a method of diagnosing malaria in a human subject, wherein the malaria is characterised by the presence of pHRT and/or pPGM comprising:
i) obtaining a biological sample from the subject;
ii) applying a monoclonal antibody specific for pHRT or pPGM, wherein the presence of pHPRT or pPGM creates an antibody-biomarker complex;
iii) applying a detection agent that detects the antibody-biomarker complex; and
iv) diagnosing malaria where the detection agent in step (iii) is detected.

According to yet another aspect of the invention, the invention provides a kit for use in determining the malarial status of a subject comprising at least one agent for determining the level of pHPRT and/or pPGM and/or one or more of the alternative biomarkers in a biological sample provided by the subject.

The agent may be an antibody

The kit may further comprise instructions for suitable operational parameters in the form of a label or separate insert.

The kit may further comprise one or more pHPRT and/or pPGM samples to be used as standard(s) for calibration and comparison.

The invention may further provide use of the level of pHPRT and/or pPGM and/or one or more of the alternative biomarkers as a biomarker to determine the malarial status of a subject.

According to another aspect the invention may provide a method of diagnosing malaria in a subject comprising:
i. obtaining a biological sample obtained from a subject;
ii. determining the presence, and optionally the level, of PF10_{—}0121 (hypoxanthine phosphoribosyltransferase, pHPRT) and/or PF11_{—}0208 phosphoglycerate mutase, pPGM) in the biological sample; and
iii. diagnosing malaria if pHPRT and/or pPGM are present in the sample.

In an alternative embodiment, in step (ii) the presence, and optionally the level, of one or more the alternative biomarkers is determined, and this is used in step (iii) to diagnose malaria.

Preferably at least the level of pHPRT is determined

According to another aspect the invention may provide a method of treating malaria in a subject comprising:
i. obtaining a biological sample obtained from a subject;
ii. determining the presence, and optionally the level, of PF10_{—}0121 (hypoxanthine phosphoribosyltransferase, pHPRT) and/or PF11_{—}0208 phosphoglycerate mutase, pPGM) in the biological sample; and
iii. administering anti-malarial drugs if pHPRT and/or pPGM are present in the sample.

In an alternative embodiment, in step (ii) the presence, and optionally the level, of one or more the alternative biomarkers is determined, and this is used in step (iii) to determine if anti-malarial drugs should be administered.

The skilled man will appreciate that preferred features of any one embodiment and/or aspect of the invention may be applied to all other embodiments and/or aspects of the invention.

There now follows by way of example only a detailed description of the present invention with reference to the accompanying drawings, in which:

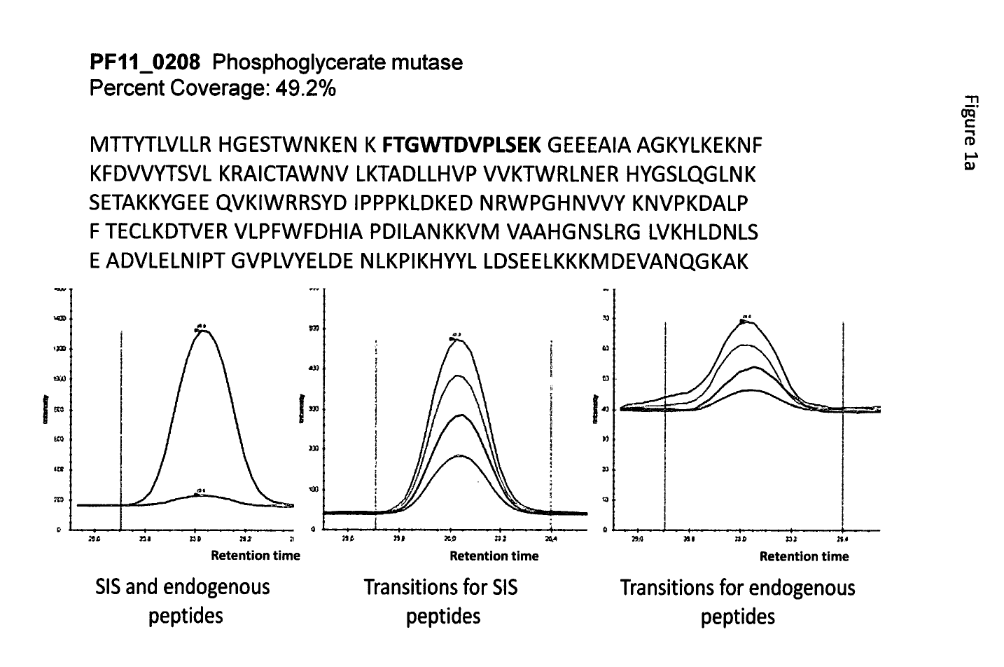

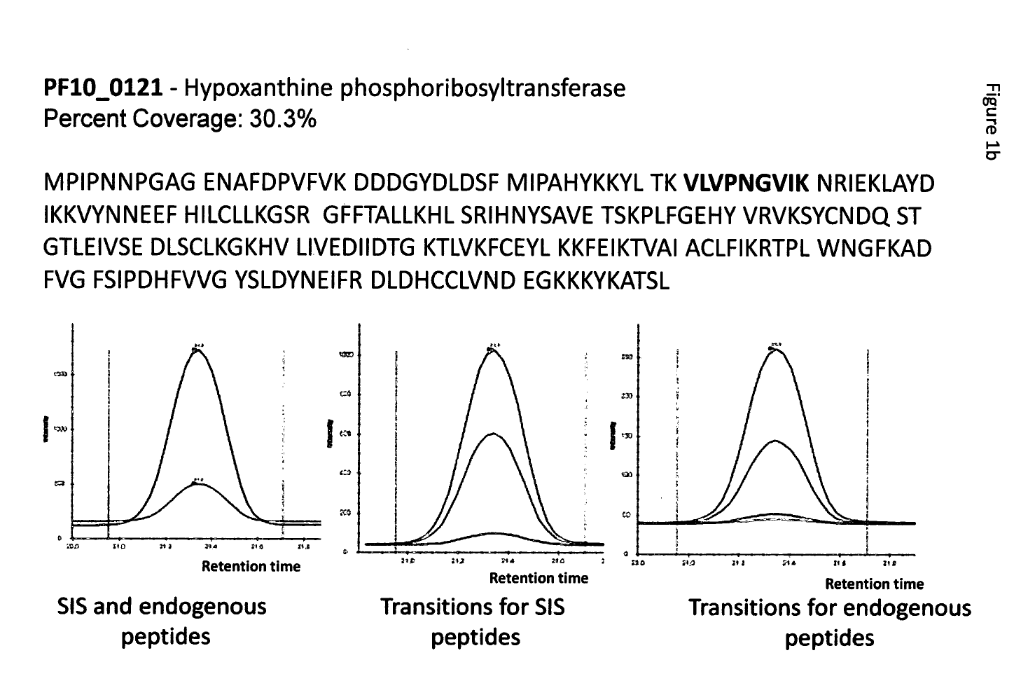

FIG. 1—details the Plasmodium falciparum proteins pPGM (FIG. 1a—Seq ID No: 1) and pHPRT (FIG. 1b—Seq ID No: 2). In each figure the top panel shows protein sequence coverage and peptide sequences used to identify parasite proteins with high confidence (highlighted and underlined). The lower panel shows a representative optimised SRM assay for selected proteotypic peptides. The left panel shows the relative abundance of heavy-labelled stable isotopic peptides (SIS) versus endogenous peptides in plasma. The middle panel shows intensity of the transitions measured for SIS peptides in plasma and the right panel shows the intensity of endogenous transitions measured in the plasma of a representative individual.

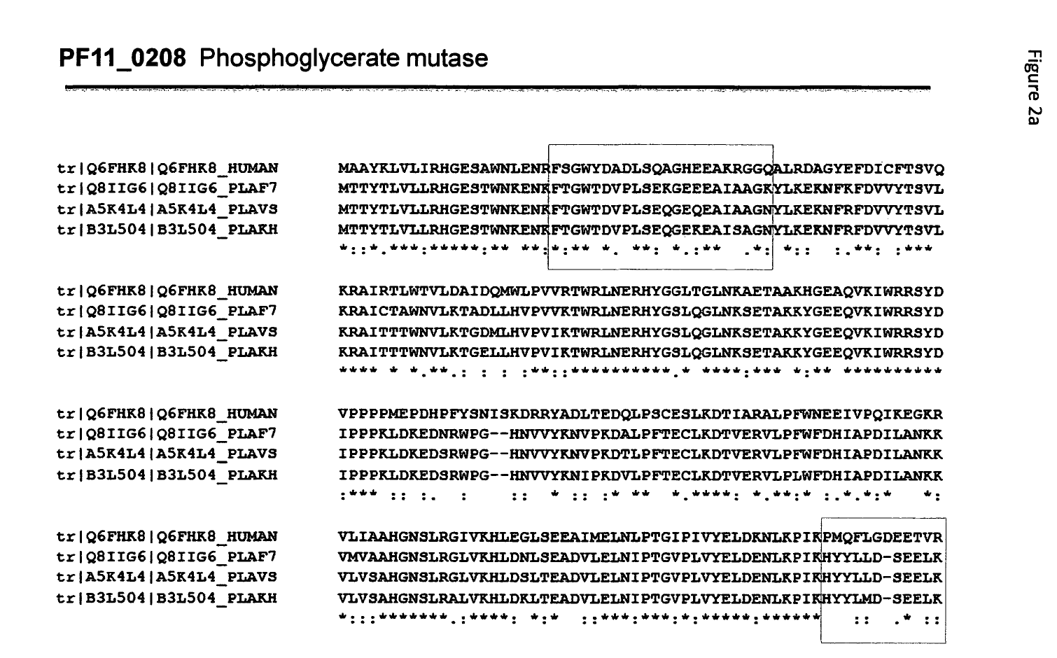

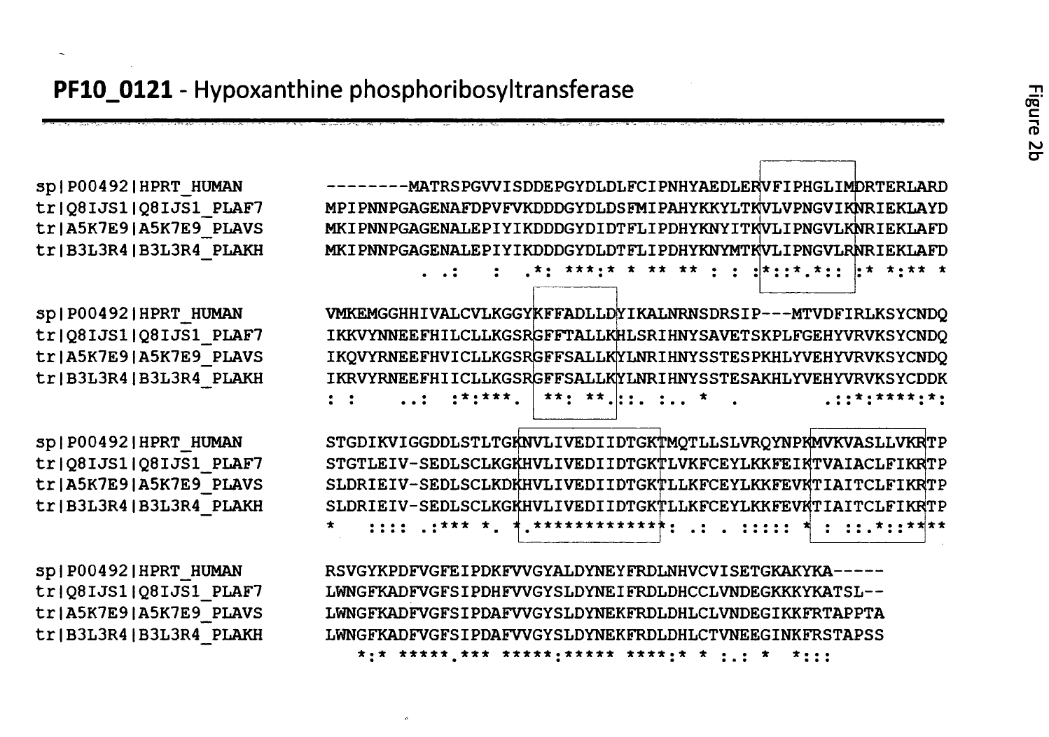

FIG. 2—shows a sequence alignment of Plasmodium falciparum proteins pPGM (FIG. 2a—Seq ID Nos: 3, 4, 5 and 6) and pHPRT (FIG. 2b—Seq ID No: 7, 8, 9 and 10) and selected proteotypic peptides and corresponding protein sequences in Homo sapiens. Data show protein alignment for parasites that may cause human malaria (P. falciparum, P. vivax, P. ovale, P. malariae and P. knowlesi) and Homo sapiens.

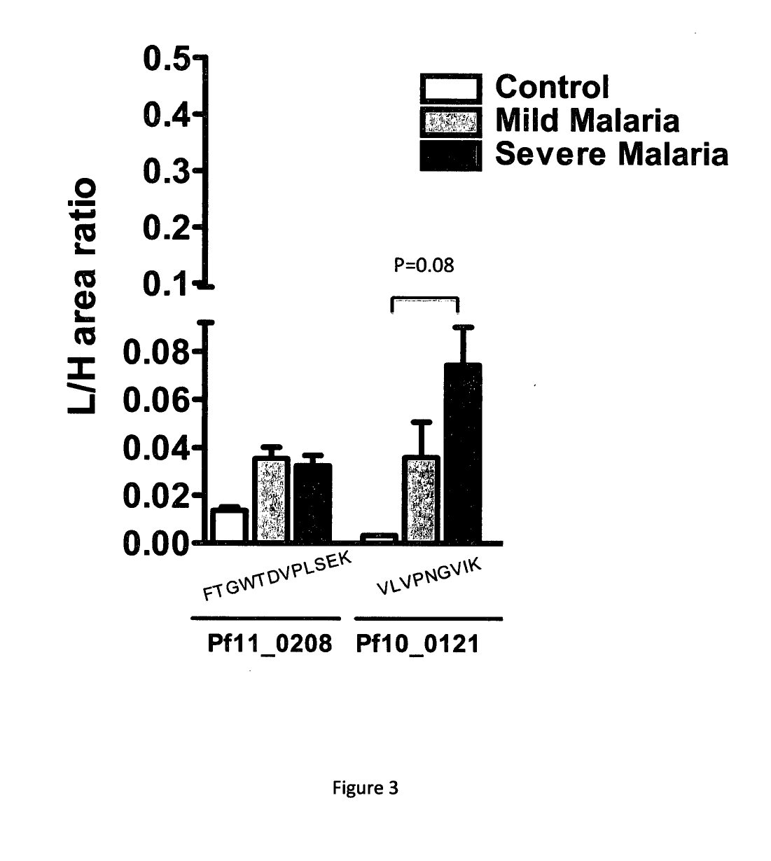

FIG. 3—illustrates the quantification of Plasmodium falciparum proteotypic peptides (PTPs) using SRM assays. Data show the mean ratio of light (endogenous) to heavy-labelled peptides (L/H area ratio) corresponding to the Plasmodium falciparum proteins pPGM and pHPRT measured in plasma samples from Gambian children with severe malaria (N=15), mild malaria (N=15) and convalescent controls (28 days after treatment) (N=15). Error bars indicate standard error of the mean. *** P<0.001, and NS: non-significant.

FIG. 4—is a table detailing P. falciparum proteins identified in plasma samples from children with severe malaria. * A score of >20 is acceptable for protein identification. The peptide sequences referred to are included in the sequence listing as Seq ID Nos: 11-87)

### METHODS

### Samples

Sample processing and storage were performed according to standard operating procedures at the MRC Laboratories in the Gambia which follow good clinical and laboratory practice (GCLP) regulations. Blood samples were processed within 2 hours of collection. Plasma was separated and stored at −80° C.

### Selective Reaction Monitoring (SRM) Validation Assays

### Sample Preparation.

Twelve microlitres of plasma samples from 48 individual patients were delipidated and depleted from the 14 most abundant proteins (MARS Human-14, Agilent Technologies), precipitated and quantified.

### Peptide Selection and Synthesis.

For each protein three to five proteotypic peptides (PTP) were selected. Based on the y-series fragment ions, three to five transitions were included for each of the two main charge states for each peptide. The predicted PTPs were synthesized (unpurified) with isotopically-labeled amino acids either with a C-term Arg (¹³C₆ ¹⁵N₄) or a C-term Lys (¹³C₆¹⁵N₂) using SPOT synthesis (NT Peptide Technology, Germany) and used as a reference to develop the corresponding SRM assays. The mass difference between the endogenous and the heavy labeled peptides for the 1+ fragment was either 8 or 10 Da. The stable isotope standard peptides were added prior to protein digestion and used as internal standards.

Purified heavy labelled peptides were synthesized on an APEX396 solid phase synthesizer (AAPPTec, Louisville, Ky., US). A 9-fluorenylmethyloxycarbonyl (Fmoc) strategy was used in the synthesis of the peptides. Initially, the first Fmoc amino acid was attached to an insoluble support resin via an acid labile linker. Deprotection of the Fmoc group was accomplished by treatment of the amino acid with 20% piperidine in dimethylformamide (DMF). The second Fmoc amino acid was coupled utilizing in situ activation of the amino acid to the growing peptide chain. After the desired peptide is synthesized, the resin bound peptide was deprotected and detached from the solid support via trifluoroacetic acid (TFA) cleavage. Peptides were precipitated with ether, dried and analysed on a reversed phase HPLC column and MALDI-TOF mass spectrometer (Ultraflex, Bruker Daltonics, Germany).

### Peptide SRM Standard Curve and Linear Range.

The peptides were weighed with high precision scale, solubilized in 10% ACN and 0.5% FA and dried in a speed vacuum centrifuge. Serial dilutions from a stock concentration of 23 ng/μL were produced for the peptides GILAADESTQTIK (Seq ID No: 88) and VLVPNGVIK (Seq ID No: 89) in 2% ACN and 0.1% FA.

SRM assays:

For SRM assays, 2 to 3 peptides and 3 to 5 transitions per peptide were used to target the pPGM or pHPRT proteins. Depleted plasma samples were spiked with a mixture of stable isotope standard (SIS) peptides in equimolar amount to get 109.1 fmol of each SIS peptide and 1 μg of plasma proteins in 1 μL. The mixture was reduced with 200 mM DTT in 100 mM Tris buffer, pH 7.8 for 30 min, alkylated with 200 mM iodoacetamide in 100 mM Tris buffer, pH 7.8 for 30 min followed by second 30 minutes incubation with DTT. The proteins were digested with sequencing grade modified trypsin (Promega) at 37° C. overnight using an enzyme to substrate ratio of 1:20. The digestion was stopped by adding 5% formic acid. Samples were desalted by Sep-Pak Light C18 cartridge (Waters), dried by speed vacuum and resuspended in buffer A (2% ACN, 0.1% FA) to get a final concentration of 1 μg/μL for plasma sample and 109.1 fmol/μL for each heavy-labelled peptide. One μL was injected on the Agilent 6460 Triple Quadrupole instrument coupled to an Agilent 1200 HPLC system equipped with a Chip-cube (Agilent Technologies).

Two separate MRM methods with 45 or 47 transitions each were used to obtain cycle times at 1.8 s and dwell-times at 35 ms. The full width at half height was 0.3 min. The optimal collision energy (CE) used for SRM assays was derived from empirical data of SIS peptide fragmentation in a 6520 Q-TOF instrument (Agilent Technologies). Q-TOF MS/MS data from SIS peptides were searched against a FASTA file that included (1) a concatenated sequence of all synthetic peptides, (2) P. falciparum 3D7 and (3) Homo sapiens proteomes to search MS/MS data.

### HPLC:

Samples were analyzed on an Agilent 1200 Series HPLC-Chip (C18 chip with 160 nL trap column) using a 0.3 μl/min flow rate and a 60 minutes gradient (as for the shotgun experiment). Briefly, we used a linear gradient from 3 to 40% buffer B (95% ACN, 0.1% FA) for 46.67 min, followed by a sharp increase to 100% within 4 min, kept for 5.33 minutes followed by a linear gradient from 100 to 3% for 4 min to finally equilibrate the system for 9 min with 3% buffer B.

### Data Analysis

SRM data were analyzed using Skyline software (v1.2) (MacLean, B., et al (2010) Skyline: an open source document editor for creating and analyzing targeted proteomics experiments. Bioinformatics 26, 966-968). Relative quantitation was done using the area ratio (light/heavy) and for each peptide only the 3 most intense transitions were considered. For each selected peptides, the most intense peak was used as quantifier and the others were used as qualifiers to validate the ratio and the retention time of the peptide measured. Retention times and peak selection was initially performed automatically by the software and validated by inspection of individual chromatograms, and selection was based on the retention time obtained with the heavy labelled peptides. The result was an alignment of the endogenous with the heavy labelled peptides. In those cases where transitions for endogenous peptides were not well resolved (eg, retention time mismatches), the analysis was discontinued.

Protein sequence alignment was performed using Clustal Omega and visualized in ClustalX v2.1. (Larkin, M. A et al (2007) Clustal W and Clustal X version 2.0. Bioinformatics 23, 2947-2948; Sievers, F., et al (2011) Fast, scalable generation of high-quality protein multiple sequence alignments using Clustal Omega. Mol Syst Biol 7, 539). The statistical analysis was done with GraphPad Prism 5 (GraphPad Software Inc, USA) and Stata 11 (StataCorp LP, USA).

### Results

Selected Reaction Monitoring Assays to Quantify P. falciparum Proteins

To validate and quantify the P. falciparum proteins pPGM and pHPRT SRM assays were used. Initially 6 proteotypic peptides were selected for pHPRT and 8 were selected for pPGM (see Table 1). SRM-based quantification was performed only for those cases where both the heavy-labeled and the endogenous peptide consistently showed identical retention times and at least 3 transitions (n=45). These criteria were met by 1 proteotypic peptide for pPGM and pHPRT.

TABLE 1

| Description | Peptides sequence |
|---|---|
| PF10_0121 hypoxanthine | DLDHCCLVNDEGK |
| phosphoribosyltransferase | Seq ID No: 90 |
| (HPRT) | HVLIVEDIIDTGK |
| | Seq ID No: 91 |
| | IHNYSAVETSKPLFGEHYVR |
| | Seq ID No: 92 |
| | GFFTALLK |
| | Seq ID No: 93 |
| | LAYDIKK |
| | Seq ID No: 94 |
| | VLVPNGVIK |
| | Seq ID No: 95 |
| PF11 0208 | HYGSLQGLNK |
| phosphoglycerate mutase | Seq ID No: 96 |
| (PGM) | FTGWTDVPLSEK |
| | Seq ID No: 97 |
| | TADLLHVPVVK |
| | Seq ID No: 98 |
| | HGESTWNKENK |
| | Seq ID No: 99 |
| | VLPFWFDHIAPDILANKK |
| | Seq ID No: 100 |
| | AICTAWNVLK |
| | Seq ID No: 101 |
| | KYGEEQVK |
| | Seq ID No: 102 |

The transitions detected for each peptide are shown in FIG. 1. The SIS and the endogenous peptides were eluted at the same retention time. In the majority of cases, the intensity of the precursor was higher for the SIS peptide. However, despite lower intensity the number of transitions detected with endogenous peptides was higher than those of spiked peptides. Peptides were detected for all the proteins and in most cases analysed. The area of the transition with the highest intensity (quantifier) of the endogenous peptide (light) and the corresponding area of the heavy-labeled peptide (spiked) were used to calculate the ratios to perform relative quantitation of the peptides in individual samples. pPGM was measured with FTGWTDVPLSEK—Seq ID No: 96 peptide and could be quantified in 36 patients, and pHPRT was measured with VLVPNGVIK—Seq ID No: 94 and could be quantified in 28 patients.

The uniqueness criterion used for PTP selection of P. falciparum proteins was based on differences with host protein sequence (Homo sapiens) but not with other parasite species that may cause human malaria. The sequence alignment indicates that peptides selected to quantify P. falciparum PGM and HPRT were species specific.

P. falciparum Protein Validation in Plasma and Clinical Correlates

Using SRM, the parasite peptides were detected mostly in plasma from severe malaria cases (N=15) but were also found in plasma samples from mild malaria (N=15) and convalescent cases (N=15). The SRM assays for pHPRT indicated a significantly higher concentration of this protein in severe malaria cases as compared to mild malaria cases. pPGM was higher in acute malaria cases compared with convalescent controls (FIG. 3).

1. A method for determining the malarial status of a subject, comprising the steps of
i providing a biological sample obtained from a subject;
iii determining the level, or presence, of PF10_{—}0121 (hypoxanthine phosphoribosyltransferase, pHPRT) and/or PF11_{—}0208 (phosphoglycerate mutase, pPGM) in the biological sample.

2. The method of claim 1 wherein the presence of pHPRT and/or pPGM is indicative/diagnostic of malaria in a subject.

3. The method of one or more preceding claim further comprising the step of comparing the level determined in step (ii) with one or more pre-determined reference values.

4. The method of one or more preceding claim wherein the presence of pHPRT is diagnostic of acute or symptomatic malaria.

5. The method of one or more preceding claim wherein the method is used for any one or more of the following: to diagnose malaria; to advise on the prognosis of a subject with malaria; to monitor disease progression; and to monitor effectiveness or response of a subject to a particular treatment.

6. A method for determining the appropriate treatment for a subject comprising the steps of:
(a) providing a biological sample obtained from a subject;
(b) determining the level, or presence, of pHPRT and/or pPGM in the biological sample from said subject; and
(c) using the results in (b) to determine the most appropriate therapy.

7. The method of claim 6 wherein the presence of pHPRT and/or pPGM in the sample indicates that the subject should be treated as having malaria and anti-malarial drugs should be administered.

8. The method of claim 6 or 7 comprising a further step of comparing the level of pHPRT and/or pPGM determined in step (b) with one or more predetermined reference values prior to step (c).

9. A method of determining the response of a subject with malaria to a particular treatment comprising the steps of:
(a) providing a biological sample obtained from a subject;
(b) determining the level of pHPRT and/or pPGM in the biological sample from said subject; and
(c) comparing the level of pHPRT and/or pPGM determined in step (b) with one or more reference values.

10. The method of claim 9 wherein the reference value is the level of pHPRT and/or pPGM in an earlier sample from the same subject.

11. A method of diagnosing malaria in a subject comprising:
i. obtaining a biological sample obtained from a subject;
ii. determining the presence, and optionally the level, of PF10_{—}0121 (hypoxanthine phosphoribosyltransferase, pHPRT) and/or PF11_{—}0208 phosphoglycerate mutase, pPGM) in the biological sample; and
iii. diagnosing malaria if pHPRT and/or pPGM are present in the sample.

12. A method of treating malaria in a subject comprising:
i. obtaining a biological sample obtained from a subject;
ii. determining the presence, and optionally the level, of PF10_{—}0121 (hypoxanthine phosphoribosyltransferase, pHPRT) and/or PF11_{—}0208 phosphoglycerate mutase, pPGM) in the biological sample; and
iii. administering anti-malarial drugs if pHPRT and/or pPGM are present in the sample.

13. The method of one or more preceding claim wherein the biological sample is a plasma sample.

14. A kit for use in determining the malarial status of a subject comprising at least one agent for determining the level of pHPRT and/or pPGM in a biological sample provided by the subject.

15. The kit of claim 14 further comprising instructions for suitable operational parameters in the form of a label or separate insert.


SEQUENCE LISTING (102 sequences)

SEQ ID NO: 1 - protein, 250 aa, Plasmodium falciparum
MTTYTLVLLRHGESTWNKENKFTGWTDVPLSEKGEEEAIAAGKYLKEKNFKFDVVYTSVLKRAICTAWNVLKTADLLHVPVVKTWRLNERHYGSLQGLNKSETAKKYGEEQVKIWRRSYDIPPPKLDKEDNRWPGHNVVYKNVPKDALPFTECLKDTVERVLPFWFDHIAPDILANKKVMVAAHGNSLRGLVKHLDNLSEADVLELNIPTGVPLVYELDENLKPIKHYYLLDSEELKKKMDEVANQGKAK

SEQ ID NO: 2 - protein, 231 aa, Plasmodium falciparum
MPIPNNPGAGENAFDPVFVKDDDGYDLDSFMIPAHYKKYLTKVLVPNGVIKNRIEKLAYDIKKVYNNEEFHILCLLKGSRGFFTALLKHLSRIHNYSAVETSKPLFGEHYVRVKSYCNDQSTGTLEIVSEDLSCLKGKHVLIVEDIIDTGKTLVKFCEYLKKFEIKTVAIACLFIKRTPLWNGFKADFVGFSIPDHFVVGYSLDYNEIFRDLDHCCLVNDEGKKKYKATSL

SEQ ID NO: 3 - protein, 54 aa, Plasmodium falciparum
FTGWTDVPLSEKGEEEAIAAGKAICTAWNVLKHYGSLQGLNKHYYLLDSEELKK

SEQ ID NO: 4 - protein, 54 aa, Plasmodium vivax
FTGWTDVPLSEQGEQEAIAAGNAITTTWNVLKHYGSLQGLNKHYYLLDSEELKK

SEQ ID NO: 5 - protein, 54 aa, Plasmodium knowlesi
FTGWTDVPLSEQGEKEAISAGNAITTTWNVLKHYGSLQGLNKHYYLMDSEELKK

SEQ ID NO: 6 - protein, 55 aa, Homo sapiens
FSGWYDADLSQAGHEEAKRGGQAIRTLWTVLDHYGGLTGLNKPMQFLGDEETVRK

SEQ ID NO: 7 - protein, 49 aa, Plasmodium falciparum
VLVPNGVIKGFFTALLKHVLIVEDIIDTGKTVAIACLFIKRTPLWNGFK

SEQ ID NO: 8 - protein, 49 aa, Plasmodium vivax
VLIPNGVLKGFFSALLKHVLIVEDIIDTGKTIAITCLFIKRTPLWNGFK

SEQ ID NO: 9 - protein, 49 aa, Plasmodium knowlesi
  misc_feature (49)..(49): Xaa can be any naturally occurring amino acid
VLIPNGVLRGFFSALLKHVLIVEDIIDTGKTIAITCLFIKRTPLWNGFX

SEQ ID NO: 10 - protein, 49 aa, Homo sapiens
VFIPHGLIMKFFADLLDNVLIVEDIIDTGKMVKVASLLVKRTPRSVGYK

SEQ ID NO: 11 - protein, 7 aa, Plasmodium falciparum
VTALNEK

SEQ ID NO: 12 - protein, 11 aa, Plasmodium falciparum
WDDIGGLDEVK

SEQ ID NO: 13 - protein, 8 aa, Plasmodium falciparum
KIHNNINK

SEQ ID NO: 14 - protein, 10 aa, Plasmodium falciparum
DSTLIMQLLR

SEQ ID NO: 15 - protein, 8 aa, Plasmodium falciparum
NLLSVAYK

SEQ ID NO: 16 - protein, 7 aa, Plasmodium falciparum
KDFETLK

SEQ ID NO: 17 - protein, 11 aa, Plasmodium falciparum
KHSEFISFPIK

SEQ ID NO: 18 - protein, 10 aa, Plasmodium falciparum
GSFGEVILSR

SEQ ID NO: 19 - protein, 16 aa, Plasmodium falciparum
ATAGDTHLGGEDFDNR

SEQ ID NO: 20 - protein, 13 aa, Plasmodium falciparum
TTPSYVAFTDTER

SEQ ID NO: 21 - protein, 11 aa, Plasmodium falciparum
GADIIVATPGR

SEQ ID NO: 22 - protein, 9 aa, Plasmodium falciparum
NAIYHATFR

SEQ ID NO: 23 - protein, 8 aa, Plasmodium falciparum
IYIHGTQK

SEQ ID NO: 24 - protein, 7 aa, Plasmodium falciparum
EDTISEK

SEQ ID NO: 25 - protein, 18 aa, Plasmodium falciparum
AAVPSGASTGIYEALELR

SEQ ID NO: 26 - protein, 14 aa, Plasmodium falciparum
INPHVLGEIAGITK

SEQ ID NO: 27 - protein, 15 aa, Plasmodium falciparum
NINDLQLQTSTQSLK

SEQ ID NO: 28 - protein, 9 aa, Plasmodium falciparum
VVESADAWR

SEQ ID NO: 29 - protein, 11 aa, Plasmodium falciparum
YETGGTLEGLR

SEQ ID NO: 30 - protein, 10 aa, Plasmodium falciparum
AICTAWNVLK

SEQ ID NO: 31 - protein, 9 aa, Plasmodium falciparum
LSSQLLEEK

SEQ ID NO: 32 - protein, 12 aa, Plasmodium falciparum
TVYDDGVIYDAK

SEQ ID NO: 33 - protein, 8 aa, Plasmodium falciparum
NILVELVK

SEQ ID NO: 34 - protein, 10 aa, Plasmodium falciparum
ASQAMPTVIK

SEQ ID NO: 35 - protein, 10 aa, Plasmodium falciparum
NTPSSSGLVK

SEQ ID NO: 36 - protein, 12 aa, Plasmodium falciparum
NVCTEAGMFAIR

SEQ ID NO: 37 - protein, 9 aa, Plasmodium falciparum
QLEQDIQLK

SEQ ID NO: 38 - protein, 9 aa, Plasmodium falciparum
TCVSQALSK

SEQ ID NO: 39 - protein, 10 aa, Plasmodium falciparum
SSLSSKPGVK

SEQ ID NO: 40 - protein, 7 aa, Plasmodium falciparum
FLNIDSK

SEQ ID NO: 41 - protein, 10 aa, Plasmodium falciparum
STTTGHIIYK

SEQ ID NO: 42 - protein, 16 aa, Plasmodium falciparum
DLVNDANVDADESGLK

SEQ ID NO: 43 - protein, 9 aa, Plasmodium falciparum
ESTLHLVLR

SEQ ID NO: 44 - protein, 13 aa, Plasmodium falciparum
IQDKEGIPPDQQR

SEQ ID NO: 45 - protein, 9 aa, Plasmodium falciparum
TLSDYNIQK

SEQ ID NO: 46 - protein, 12 aa, Plasmodium falciparum
NLVPGESVYGEK

SEQ ID NO: 47 - protein, 8 aa, Plasmodium falciparum
LTYEQIEK

SEQ ID NO: 48 - protein, 13 aa, Plasmodium falciparum
AHNEFISNLPNIK

SEQ ID NO: 49 - protein, 20 aa, Plasmodium falciparum
IVHDNPDTNDEDINMQNLFK

SEQ ID NO: 50 - protein, 15 aa, Plasmodium falciparum
EENSNYPHELLNTIK

SEQ ID NO: 51 - protein, 15 aa, Plasmodium falciparum
VLSGEFFVVNPHLLK

SEQ ID NO: 52 - protein, 12 aa, Plasmodium falciparum
TATPEQAQLVHK

SEQ ID NO: 53 - protein, 9 aa, Plasmodium falciparum
NIAQPIINR

SEQ ID NO: 54 - protein, 8 aa, Plasmodium falciparum
VHIEYGEK

SEQ ID NO: 55 - protein, 7 aa, Plasmodium falciparum
HYLIHLR

SEQ ID NO: 56 - protein, 12 aa, Plasmodium falciparum
AQFMGDSELAGR

SEQ ID NO: 57 - protein, 8 aa, Plasmodium falciparum
KVSVFAEK

SEQ ID NO: 58 - protein, 10 aa, Plasmodium falciparum
YEEVALEIKK

SEQ ID NO: 59 - protein, 12 aa, Plasmodium falciparum
GILLSGEPGTGK

SEQ ID NO: 60 - protein, 10 aa, Plasmodium falciparum
EALGVALDAR

SEQ ID NO: 61 - protein, 9 aa, Plasmodium falciparum
KGIVNIETK

SEQ ID NO: 62 - protein, 14 aa, Plasmodium falciparum
ATIAGGGVIPHIHK

SEQ ID NO: 63 - protein, 12 aa, Plasmodium falciparum
TLIPGYASYTNK

SEQ ID NO: 64 - protein, 9 aa, Plasmodium falciparum
LGQSMEAVR

SEQ ID NO: 65 - protein, 13 aa, Plasmodium falciparum
AGSCGSLQPDLIK

SEQ ID NO: 66 - protein, 13 aa, Plasmodium falciparum
VVCDSYVDLAYNR

SEQ ID NO: 67 - protein, 11 aa, Plasmodium falciparum
IILIGDSGVGK

SEQ ID NO: 68 - protein, 9 aa, Plasmodium falciparum
NNNTHDLIK

SEQ ID NO: 69 - protein, 13 aa, Plasmodium falciparum
INFLVPGNQGVVK

SEQ ID NO: 70 - protein, 13 aa, Plasmodium falciparum
INFLVPGNQGVVK

SEQ ID NO: 71 - protein, 10 aa, Plasmodium falciparum
LVAVEGVVNK

SEQ ID NO: 72 - protein, 14 aa, Plasmodium falciparum
EDIEIESSHMPHFK

SEQ ID NO: 73 - protein, 11 aa, Plasmodium falciparum
VNNNLLNSIEK

SEQ ID NO: 74 - protein, 12 aa, Plasmodium falciparum
IDEIVLVGGSTR

SEQ ID NO: 75 - protein, 15 aa, Plasmodium falciparum
HKYNTNTDSNNIDIK

SEQ ID NO: 76 - protein, 8 aa, Plasmodium falciparum
QIPYTMAK

SEQ ID NO: 77 - protein, 8 aa, Plasmodium falciparum
MIGEAWNK

SEQ ID NO: 78 - protein, 10 aa, Plasmodium falciparum
SNFIQEIHKK

SEQ ID NO: 79 - protein, 8 aa, Plasmodium falciparum
IYLEDNIK

SEQ ID NO: 80 - protein, 8 aa, Plasmodium falciparum
EVNTPFFK

SEQ ID NO: 81 - protein, 9 aa, Plasmodium falciparum
YSSLNDIDK

SEQ ID NO: 82 - protein, 11 aa, Plasmodium falciparum
IWHHTFYNELR

SEQ ID NO: 83 - protein, 11 aa, Plasmodium falciparum
IWHHTFYNELR

SEQ ID NO: 84 - protein, 7 aa, Plasmodium falciparum
RGILTLK

SEQ ID NO: 85 - protein, 10 aa, Plasmodium falciparum
GHILEVDIKK

SEQ ID NO: 86 - protein, 11 aa, Plasmodium falciparum
IGGIGTVPVGR

SEQ ID NO: 87 - protein, 7 aa, Plasmodium falciparum
TIEKFEK

SEQ ID NO: 88 - protein, 13 aa, Plasmodium falciparum
GILAADESTQTIK

SEQ ID NO: 89 - protein, 9 aa, Plasmodium falciparum
VLVPNGVIK

SEQ ID NO: 90 - protein, 13 aa, Plasmodium falciparum
DLDHCCLVNDEGK

SEQ ID NO: 91 - protein, 13 aa, Plasmodium falciparum
HVLIVEDIIDTGK

SEQ ID NO: 92 - protein, 20 aa, Plasmodium falciparum
IHNYSAVETSKPLFGEHYVR

SEQ ID NO: 93 - protein, 8 aa, Plasmodium falciparum
GFFTALLK

SEQ ID NO: 94 - protein, 7 aa, Plasmodium falciparum
LAYDIKK

SEQ ID NO: 95 - protein, 9 aa, Plasmodium falciparum
VLVPNGVIK

SEQ ID NO: 96 - protein, 10 aa, Plasmodium falciparum
HYGSLQGLNK

SEQ ID NO: 97 - protein, 12 aa, Plasmodium falciparum
FTGWTDVPLSEK

SEQ ID NO: 98 - protein, 11 aa, Plasmodium falciparum
TADLLHVPVVK

SEQ ID NO: 99 - protein, 11 aa, Plasmodium falciparum
HGESTWNKENK

SEQ ID NO: 100 - protein, 18 aa, Plasmodium falciparum
VLPFWFDHIAPDILANKK

SEQ ID NO: 101 - protein, 10 aa, Plasmodium falciparum
AICTAWNVLK

SEQ ID NO: 102 - protein, 8 aa, Plasmodium falciparum
KYGEEQVK